(19) United States
(12) Patent Application Publication
(10) Pub. No.: US 2019/0298735 A1
(43) Pub. Date: Oct. 3, 2019

(54) TREATMENT AND PREVENTION OF CLOSTRIDIUM DIFFICILE COLITIS USING MISOPROSTOL

(71) Applicant: Vanderbilt University, Nashville, TN (US)

(72) Inventor: David ARONOFF, Brentwood, TN (US)

(21) Appl. No.: 16/316,067

(22) PCT Filed: Jul. 7, 2017
(86) PCT No.: PCT/US17/41090

§ 371 Date: Jan. 8, 2019

Related U.S. Application Data
(60) Provisional application No. 62/359,926, filed on Jul. 8, 2016.

(51) Int. Cl. A61K 31/5575; A61K 9/20; A61K 38/14; A61K 35/741; A61K 35/745; A61K 35/747; A61P 31/04

(52) U.S. Cl. CPC A61K 31/5575; A61K 9/20; A61K 38/14; A61K 2035/115; A61K 35/745; A61K 35/747; A61P 31/04; A61K 35/741

(57) ABSTRACT

The present invention relates to compositions and methods for the treatment or prevention of the recurrence of C. difficile infections using misoprostol.

### CROSS REFERENCE TO RELATED APPLICATIONS

This application claims the benefit of U.S. Provisional Patent Application Ser. No. 62/359,926, filed Jul. 8, 2016, the disclosure of which is expressly incorporated herein by reference.

### STATEMENT REGARDING FEDERALLY SPONSORED RESEARCH

This invention was made with Government Support under Grant No. DK058404 awarded by the National Institutes of Health. The Government has certain rights to the invention.

### FIELD

The present disclosure relates to compositions and methods for the treatment or prevention of the recurrence of C. difficile infections using misoprostol.

### BACKGROUND

Clostridium difficile infection (CDI) is a leading nosocomial infection and the primary identifiable cause of antibiotic-associated diarrhea (AAD). It can lead to multiple CDI recurrences, sepsis, toxic megacolon, and death. Recurrent CDI complicates 20% of primary episodes of disease and is associated with an increased risk of death and a poor quality of life. While probiotics or fecal transplantation have met with some success in breaking the cycle of recurrent CDI, there remains a need for simple, safe, and cost-effective solutions to this problem.

The compositions and methods disclosed herein address these and other needs.

### SUMMARY

The inventors have found that misoprostol treatment can reduce the severity of Clostridium difficile infection (CDI). Thus, in addition to the FDA approved use for prevention and treatment of nonsteroidal anti-inflammatory drugs (NSAID)-induced gastric ulcers, misoprostol can also be used to treat C. difficile infections and its associated symptoms. In some embodiments, the use of misoprostol can be used to prevent or treat recurrent C. difficile infections, alleviating the need for treatments with harsh antibiotics or fecal transplants.

In one aspect, disclosed herein is a method for treating or preventing the recurrence of a C. difficile infection comprising administering to a host in need thereof an effective amount of misoprostol, or a pharmaceutically acceptable salt thereof.

In another aspect, provided herein is a method for treating or preventing the recurrence of a C. difficile infection comprising administering to a host in need thereof an effective amount of misoprostol, or a pharmaceutically acceptable salt thereof, and an antibiotic.

In another aspect, provided herein is a method for treating or preventing the recurrence of a C. difficile infection comprising administering to a host in need thereof an effective amount of misoprostol, or a pharmaceutically acceptable salt thereof, and vancomycin.

In one aspect, disclosed herein is a method for treating or preventing the recurrence of a C. difficile infection comprising administering to a host in need thereof an effective amount of misoprostol, or a pharmaceutically acceptable salt thereof, and a probiotic.

In another aspect, provided herein is a composition comprising misoprostol, or a pharmaceutically acceptable salt thereof, and an antibiotic.

In one aspect, provided herein is a composition comprising misoprostol, or a pharmaceutically acceptable salt thereof, and a probiotic.

In one aspect, disclosed herein is a method for reducing the severity of C. difficile colitis comprising administering to a host in need thereof an effective amount of misoprostol, or a pharmaceutically acceptable salt thereof.

In one aspect, disclosed herein is a method for reducing the severity of NSAID-associated C. difficile colitis comprising administering to a host in need thereof an effective amount of misoprostol, or a pharmaceutically acceptable salt thereof.

In one aspect, disclosed herein is a method for reducing the severity of C. difficile colitis comprising administering to a host in need thereof an effective amount of misoprostol, or a pharmaceutically acceptable salt thereof, wherein the severity of at least one symptom of C. difficile colitis is reduced.

In some embodiments, the symptoms of C. difficile colitis that can be alleviated by misoprostol include, for example, diarrhea, severe abdominal pain, loss of appetite, fever, blood or pus in the stool, and/or weight loss.

### BRIEF DESCRIPTION OF THE DRAWINGS

The accompanying figures, which are incorporated in and constitute a part of this specification, illustrate several aspects described below.

FIG. 1. NSAID administration timeline. C57BL/6 mice were treated with cefoperazone×5 days followed by 2 days of recovery and then challenged by gavage with 1×10⁴ spores of the NAP1 C. difficile strain M7404. In (A) animals received 10 mg/kg of indomethacin (INDO) by gavage daily for 3 doses as indicated by the green box. In (B) mice received 10 mg/kg/d of INDO gavage for 2 days as indicated (no dose given on day 0). In (C) mice received ibuprofen (IBUP) 5 mg/kg in drinking water for 7 days prior to inoculation. These panels correspond to FIG. 2.

FIG. 2. NSAIDs worsen CDI in mice. C57BL/6 mice were infected with C. difficile as noted in the FIG. 1 description. The NSAIDs administered were indomethacin (A & B) or ibuprofen (C). Mice were monitored for survival. n=5-10 mice per group. *P<0.05, **P<0.01 by Log-rank (Mantel-Cox) test.

FIG. 3. Indomethacin inhibits PGE2 production and worsens colitis during CDI. (A) Mice were infected with C. difficile and treated with indomethacin by gavage once daily starting on the day of infection until mice were euthanized on day +4. Tissue PGE2 concentrations were measured by ELISA. n=5 mice per group. (B) Histological assessment of colitis was performed in a blinded fashion. The score incorporates edema, neutrophilic inflammation, and epithelial damage. (C and D) Tissue cytokine and chemokine concentrations were measured by ELISA. n=5 mice per group. *P<0.05.

FIG. 4. Indomethacin alters gut microbiome. Mice were treated with indomethacin 10 mg/kg/d by gavage for 2 days, then euthanized. Cecal contents were analyzed for bacterial diversity through 16S rRNA sequencing. N=5 mice per group. Diversity represented as inverse Simpson index. *P<0.05 by t-test.

FIG. 5. Misoprostol reduces CDI severity. (A) Mice were infected by gavage with 10⁶ spores of the NAP1/BI/027 strain M7404 and treated daily with the PGE analogue misoprostol (or vehicle) by intraperitoneal (IP) injection. n=5 per group. (B) To assess intestinal permeability mice were infected with 1×10 spores of M7404 and given misoprostol 20 μg/mouse by IP injection 30 min before infection, 24 hr post-infection and at the time of FITC-dextran treatment. 2 d post infection mice were gavaged with FITC-dextran or vehicle control, then euthanized 4 hr later and concentrations of FITC-dextran in plasma were determined (*P<0.05). (C) Colonization levels of C. difficile strain 630 in the cecum for infected mice exposed to misoprostol or vehicle 96 hr post infection (*P<0.05).

FIG. 6. Misoprostol improves CDI in indomethacin-exposed mice. Female C57BL/6 mice were treated with cefoperazone for 5 days following 2 days of indomethacin (INDO) 10 mg/kg by gavage on days −2 and −1 prior to inoculation with 1×10⁴ spores of strain M7404 by gavage. In (A) mice received 20 mg misoprostol (MISO) by intraperitoneal (i.p.) injection (or vehicle) daily starting on the day of inoculation. N=7-8 mice per group. N.s., not statistically significant by Log-rank (Mantel-Cox) test. In (B) the weights of infected mice (treated with or without MISO) were recorded daily and graphed relative to baseline (day 0). *P<0.05, ***P<0.001 by unpaired t test at each time point. In (C) mice (n=7-8 per group) received MISO by i.p. injection (or vehicle) daily and stools were scored for severity of diarrhea on a 4 point scale (1—normal, 2—soft stool/discolored, 3—wet stained tail/mucous, 4—liquid/no stool). ***P<0.001 by unpaired t test at each time point.

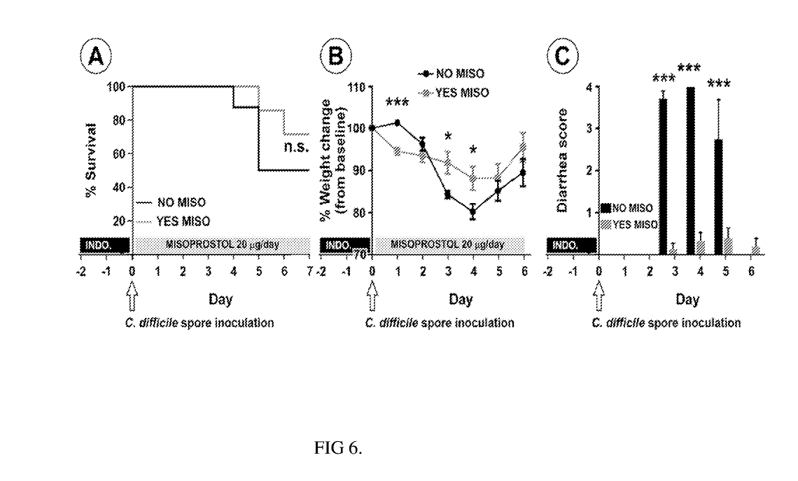

### DETAILED DESCRIPTION

The inventors have found that misoprostol treatment can reduce the severity of C. difficile infection. Thus, in addition to the currently FDA approved use for prevention and treatment of gastric ulcers, misoprostol can also be used to treat C. difficile infections. In some embodiments, the use of misoprostol can be used to treat recurrent C. difficile infections, alleviating the need for treatments with harsh antibiotics or fecal transplants. Further disclosed are dosing regimens for treatment of C. difficile infections with misoprostol alone or in combination with additional therapeutics.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this invention belongs. The following definitions are provided for the full understanding of terms used in this specification.

### Terminology

As used in the specification and claims, the singular form “a,” “an,” and “the” include plural references unless the context clearly dictates otherwise. For example, the term “a cell” includes a plurality of cells, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject.

The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

As used herein, the term “mixture” can include solutions in which the components of the mixture are completely miscible, as well as suspensions and emulsions, in which the components of the mixture are not completely miscible.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human. In some embodiments, the pharmacokinetic profiles of the systems of the present invention are similar for male and female subjects.

As used herein, the term “controlled-release” or “controlled-release drug delivery” or “extended release” refers to release or administration of a drug from a given dosage form in a controlled fashion in order to achieve the desired pharmacokinetic profile in vivo. An aspect of “controlled” drug delivery is the ability to manipulate the formulation and/or dosage form in order to establish the desired kinetics of drug release.

The phrases “concurrent administration”, “administration in combination”, “simultaneous administration” or “administered simultaneously” as used herein, means that the compounds are administered at the same point in time or immediately following one another. In the latter case, the two compounds are administered at times sufficiently close that the results observed are indistinguishable from those achieved when the compounds are administered at the same point in time.

### Misoprostol

Misoprostol is currently used as an oral antiulcer drug (Physicians Desk Reference, PDR, ed. Medical Economics Data Production Company, Montvale, N.J., 48^{th} edition, 1994, 2197-2199), specifically, misoprostol is administered for prevention of gastric ulcer in patients concurrently taking non-steroidal anti-inflammatory drugs. It is available in European countries and in the U.S. from Searle Company under the commercial name Cytotec®.

Misoprostol is a synthetic prostaglandin belonging to the E₁ series (PGE₁ analogs). Synthesis of misoprostol is described by: P. W. Collins, et al., Belgian Patent 827,127; and U.S. Pat. No. 3,965,143; see also The Merck Index, ed. Merck & Co., Inc., 11^{th} edition, 1989, 6128. The chemical name for misoprostol is (11a, 13E)-(±)-11,16-dihydroxy-16-methyl-9-oxoprost-13-en-1-oic acid methyl ester, or (±)-(methyl)-(1R,2R,3R)-3-hydroxy-2-[(E)-(4RS)-4-hydroxy-4-methyl-1-octenyl]-5-oxocyclopentaneheptanoate, or (±)-15-deoxy-(16RS)-16-hydroxy-16-methyl-PGE₁ methyl ester. The empirical formula is C₂₂H₃₈O₅. The structural formula is shown below:

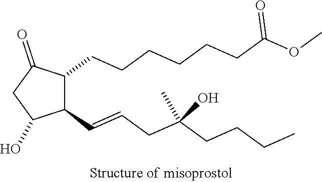 [SMILES: CCCC[C@](C)(O)C/C=C/[C@H]1[C@H](O)CC(=O)[C@@H]1CCCCCCC(=O)OC]

Four steroisomers can be found in about equal proportions (the (+) and (−) enantiomers of the 16-R- and 16-S-forms; Merck Index, 11^{th} edition, 1989, 6128). In contrast to other prostaglandins of group E and especially alprostadil, misoprostol bears a methyl group (—CH₃) on the carbon atom at position 16, In one aspect, disclosed herein is a method for treating or preventing the recurrence of a C. difficile infection comprising administering to a host in need thereof an effective amount of misoprostol, or a pharmaceutically acceptable salt thereof.

In one embodiment, the misoprostol is administered two times daily for two weeks. In one embodiment, the misoprostol is administered one time daily for two weeks. In one embodiment, the misoprostol is administered three times daily for two weeks. In one embodiment, the misoprostol is administered four times daily for two weeks.

In one embodiment, the misoprostol is administered for at least one week (for example, at least one week, at least two weeks, at least three weeks, at least four weeks, at least five weeks, at least six weeks, at least seven weeks, at least eight weeks, at least nine weeks, at least ten weeks, at least eleven weeks, or at least twelve weeks).

In one embodiment, the misoprostol is administered for one week. In one embodiment, the misoprostol is administered for two weeks. In one embodiment, the misoprostol is administered for three weeks. In one embodiment, the misoprostol is administered for four weeks. In one embodiment, the misoprostol is administered for five weeks. In one embodiment, the misoprostol is administered for six weeks. In one embodiment, the misoprostol is administered for seven weeks. In one embodiment, the misoprostol is administered for eight weeks. In one embodiment, the misoprostol is administered for nine weeks. In one embodiment, the misoprostol is administered for ten weeks. In one embodiment, the misoprostol is administered for eleven weeks. In one embodiment, the misoprostol is administered for twelve weeks. In one embodiment, the misoprostol is administered continuously. In one embodiment, the misoprostol is administered continuously in a once-daily dose.

In one embodiment, the misoprostol is in a tablet dosage form. In one embodiment, the misoprostol is comprised in an extended release dosage form. In one embodiment, the misoprostol is released directly to the colon.

In some embodiments, the dose of misoprostol comprises a range from 10 μg to 1000 μg. In some embodiments, the dose of misoprostol comprises a range from 50 g to 500 μg. In some embodiments, the dose of misoprostol comprises a range from 100 μg to 200 μg. In one embodiment, the dose of misoprostol is about 10 μg, about 25 μg, about 50 μg, about 75 μg, about 100 μg, about 125 μg, about 150 μg, about 175 μg, about 200 μg, about 250 μg, about 300 μg, about 350 g, about 400 μg, about 450 μg, about 500 μg, about 600 μg, about 700 μg, about 800 μg, about 900 μg, about 1000 μg, or more.

In some embodiments, the dose of misoprostol is 200 μg. In some embodiments, the dose of misoprostol is 50 μg. In some embodiments, the dose of misoprostol is 100 μg. In some embodiments, the dose of misoprostol is 150 μg. In some embodiments, the dose of misoprostol is 250 μg. In some embodiments, the dose of misoprostol is 300 μg. In some embodiments, the dose of misoprostol is 350 μg. In some embodiments, the dose of misoprostol is 400 μg.

In some embodiments, the daily dosage of misoprostol is 100 μg. In some embodiments, the daily dosage of misoprostol is 200 μg. In some embodiments, the daily dosage of misoprostol is 300 μg. In some embodiments, the daily dosage of misoprostol is 400 μg. In some embodiments, the daily dosage of misoprostol is 600 μg. In some embodiments, the daily dosage of misoprostol is 800 μg. In some embodiments, the daily dosage of misoprostol is 1000 μg.

In some embodiments, the active compound (for example, misoprostol) is taken 1, 2, 3, 4, 5, 6, 7, 8, or more times daily. In one embodiment, the misoprostol is administered one time daily. In one embodiment, the misoprostol is administered two times daily. In one embodiment, the misoprostol is administered three times daily. In one embodiment, the misoprostol is administered four times daily. In one embodiment, the misoprostol is administered five times daily. In one embodiment, the misoprostol is administered six times daily. In one embodiment, the misoprostol is administered seven times daily. In one embodiment, the misoprostol is administered eight times daily.

In some embodiments, the misoprostol therapy begins on day 7-10 of antibiotic therapy, after resolution of diarrhea. In some embodiments, the misoprostol is administered concurrently with vancomycin during the last four days of antibiotic therapy.

In some embodiments, the misoprostol therapy begins on day 7-10 of vancomycin therapy, after resolution of diarrhea. In some embodiments, the misoprostol is administered concurrently with vancomycin during the last four days of vancomycin therapy.

In some embodiments, the antibiotic is administered concurrently with the misoprostol for at least one day. In some embodiments, the antibiotic is administered concurrently with the misoprostol for at least two days. In some embodiments, the antibiotic is administered concurrently with the misoprostol for at least three days. In some embodiments, the antibiotic is administered concurrently with the misoprostol for at least four days. In some embodiments, the antibiotic is administered concurrently with the misoprostol for at least five days. In some embodiments, the antibiotic is administered concurrently with the misoprostol for four days.

In some embodiments, vancomycin is administered concurrently with the misoprostol for at least one day. In some embodiments, vancomycin is administered concurrently with the misoprostol for at least two days. In some embodiments, vancomycin is administered concurrently with the misoprostol for at least three days. In some embodiments, vancomycin is administered concurrently with the misoprostol for at least four days. In some embodiments, vancomycin is administered concurrently with the misoprostol for at least five days. In some embodiments, vancomycin is administered concurrently with the misoprostol for four days.

In some embodiments, the active compound (for example, misoprostol) is taken for about 1 day, about 2 days, about 3 days, about 4 days, about 5 days, about 6 days, about 7 days, about 8 days, about 9 days, about 10 days, about 14 days, about 21 days, about 28 days, or more days.

In some embodiments, the misoprostol is administered in combination with vancomycin.

In some embodiments, the misoprostol is administered concurrently with vancomycin. In some embodiments, the misoprostol is administered after vancomycin. In some embodiments, the misoprostol is administered concurrently with vancomycin for at least some of the time that vancomycin is being administered.

In some embodiments, the misoprostol is administered concurrently with vancomycin for at least one day (for example, at least one day, at least two days, at least three days, at least four days, at least five days, at least six days, at least seven days, at least eight days, at least nine days, at least ten days). In some embodiments, the misoprostol is administered concurrently with vancomycin during at least one day of the last four days of vancomycin treatment.

In some embodiments, the host or subject is a human. In some embodiments, the host or subject is a human over the age of 50.

Clostridium difficile Colitis

Clostridium difficile is a Gram-positive, anaerobic, spore-forming bacillus that was first identified in 1978 as the predominate bacterial cause of antibiotic-associated diarrhea (AAD) and pseudomembranous colitis (PMC). While the pathogenicity of C. difficile toward humans was discovered in relation to its ability to cause antibiotic-associated diarrhea and PMC, it is now known that the manifestations of C. difficile infection (CDI) can range from asymptomatic colonization, to mild diarrheal illness, to more severe disease, including PMC, toxic megacolon, sepsis, and death.

In the past 10-15 years, CDI has reemerged as an increasingly important infectious disease worldwide. The dramatic increase in the incidence of CDI, coupled with an increasingly vulnerable healthcare population has resulted in more frequent medical and surgical complications, added health care costs, and greater mortality. Furthermore, CDI is now increasingly being recognized in patient populations previously considered to be at low risk.

After steady increases over the past decade, the number of CDI-related hospital stays appears to have plateaued in recent years. It is not entirely clear if the decreased incidence is due to the success of expanded prevention and control efforts, changes in the prevalence of epidemic strains, or perhaps a combination of factors. Emerging risk factors and disease recurrence represent continued challenges in the management of CDI. The acute-care direct costs of CDI were estimated to be $4.8 billion in 2008. However, the actual cost is likely higher when considering indirect costs associated with the overall management, including treating recurrences.

In one aspect, disclosed herein is a method for reducing the severity of C. difficile colitis comprising administering to a host in need thereof an effective amount of misoprostol, or a pharmaceutically acceptable salt thereof.

In one aspect, disclosed herein is a method for reducing the severity of NSAID-associated C. difficile colitis comprising administering to a host in need thereof an effective amount of misoprostol, or a pharmaceutically acceptable salt thereof.

In one aspect, disclosed herein is a method for reducing the severity of C. difficile colitis comprising administering to a host in need thereof an effective amount of misoprostol, or a pharmaceutically acceptable salt thereof, wherein the severity of at least one symptom of C. difficile colitis is reduced.

In some embodiments, the symptoms of C. difficile colitis that can be alleviated by misoprostol include, for example, diarrhea, severe abdominal pain, loss of appetite, fever, blood or pus in the stool, and/or weight loss.

In one aspect, disclosed herein is a method for treating C. difficile-associated diarrhea comprising administering to a host in need thereof an effective amount of misoprostol, or a pharmaceutically acceptable salt thereof.

In some embodiments, the host is being treated with a non-steroidal anti-inflammatory drug (NSAIDs). Non-limiting examples of NSAIDs include, for examples, ibuprofen, naproxen, aspirin, diclofenac, ketoprofen, indomethacin, acetaminophen, celecoxib, salicylic acid, diflunisal, etodolac, fenoprofen, flurbiprofen, meclofenamic acid, mefenamic acid, flufenamic acid, toflenamic acid, meloxicam, nabumetone, oxaprozin, piroxicam, refocoxib, parecoxib, lumiracoxib, firocoxib, valdecoxib, etoricoxib, nimesulide, clonixin, salsalate, sulindac, tolmetin, valdecoxib, dexibuprofen, loxoprofen, dexketoprofen, acelofenac, ketorolac, droxicam, phenylbutazone, lornoxicam, tenoxicam, isoxicam, licofelone, and pharmaceutically acceptable salts thereof. In one embodiment, the NSAID is ibuprofen.

Combination Therapy with Antibiotics and Additional Therapeutic Agents

Current therapy for antibiotic-associated diarrhea (AAD) or CDI includes discontinuation of implicated antimicrobial or chemotherapy agents, nonspecific supportive measures, and treatment with antibiotics directed against C. difficile. Treatment of CDI with antibiotics is associated with clinical relapse of the disease. Frequency of relapse is reported to be 5-50%, with, a 20-30% recurrence rate being the most commonly quoted figure. Relapse occurs with nearly equal frequency regardless of the drug, dose, or duration of primary treatment with any of the antibiotics listed above. The major challenge in therapy is in the management of patients with multiple relapses, where antibiotic control is problematic.

The two most commonly utilized therapies are vancomycin and metronidazole, though vancoimycin is the only drug approved by the FDA for this indication. However, vancomycin is not recommended for first-line treatment of CDI mainly because it is the only antibiotic active against some serious life-threatening multi-drug resistant bacteria. Therefore, in an effort to minimize the emergence of vancomycin-resistant Enterococcus (VRE) or vancomycin-resistant Staphylococcus aureus (VRSA), the medical community discourages the use of this drug except when absolutely necessary.

Metronidazole is recommended as initial therapy out of concern for the promotion and selection of vancomycin resistant gut flora, especially enterococci. Despite reports that the frequency of C. difficile resistance may be >6% in some countries, metronidazole remains nearly as effective as vancomycin, is considerably less expensive, and can be used either orally or intravenously. Metronidazole is associated with significant adverse effects including nausea, neuropathy, leukopenia, seizures, and a toxic reaction to alcohol. Furthermore, it is not safe for use in children or pregnant women.

Although both agents are effective in treating the infection, increasing rates of treatment failures and recurrence of diarrhea in approximately 20% of patients that initially respond are deficiencies of standard therapies. Therapy with metronidazole has been reported to be an important risk factor for VRE colonization and infection. In addition, the current treatment regime is rather cumbersome, requiring up to 500 mg qid for 10 to 14 days. Thus, there is a need for better treatment for cases of CDI as well as for cases of other AAD and AAC.

Non-limiting examples of antibiotics for use in combination with misoprostol for the treatment or prevention of C. difficile infections include, but are not limited to: fidaxomicin, metronidazole, vancomycin, cadazolid, CRS3123, SMT19969, surotomycin, teicoplanin, tigecycline, ramoplanin, NVB302, nitazoxanide.

In additional embodiments, monoclonal or recombinant antibodies directed against C. difficile toxins can be used in combination with misoprostol for the treatment of prevention of C. difficile infections. (See McFarland L V. Therapies on the horizon for Clostridium difficile infections. Expert opinion on investigational drugs. 2016; 25(5):541-55). In additional embodiments, bezlotoxumab can be used in combination with misoprostol for the treatment of prevention of C. difficile infections.

In some embodiments, disclosed herein are compositions comprising misoprostol, or a pharmaceutically acceptable salt thereof, and an antibiotic.

In one aspect, provided herein is a method for treating or preventing the recurrence of a Clostridium difficile infection comprising administering to a host in need thereof an effective amount of misoprostol, or a pharmaceutically acceptable salt thereof, and an antibiotic.

In one embodiment, the antibiotic is selected from the group consisting of fidaxomicin, metronidazole, vancomycin, cadazolid, CRS3123, SMT19969, surotomycin, teicoplanin, tigecycline, ramoplanin, NVB302, ridinilazole, miconazole nitrate, rifalazil, tolevamer, and nitazoxanide.

In one embodiment, the antibiotic is vancomycin. In one embodiment, the antibiotic is metronidizaole. In one embodiment, the antibiotic is fidaxomicin. In one embodiment, the antibiotic is cadazolid. In one embodiment, the antibiotic is CRS3123. In one embodiment, the antibiotic is SMT19969. In one embodiment, the antibiotic is surotomycin. In one embodiment, the antibiotic is teicoplanin. In one embodiment, the antibiotic is tigecycline. In one embodiment, the antibiotic is ramoplanin. In one embodiment, the antibiotic is NVB302. In one embodiment, the antibiotic is nitazoxanide.

In one embodiment, the antibiotic is administered prior to the misoprostol. In one embodiment, the antibiotic is administered concurrently with the misoprostol. In one embodiment, the antibiotic is administered after the misoprostol.

In one embodiment, disclosed herein is a composition comprising misoprostol, or a pharmaceutically acceptable salt thereof, and vancomycin. In one embodiment, disclosed herein is a method for treating or preventing the recurrence of a C. difficile infection comprising administering to a host in need thereof an effective amount of misoprostol, or a pharmaceutically acceptable salt thereof, and vancomycin.

In some embodiments, the misoprostol and the antibiotic are combined in a single dosage form (for example, in a single oral tablet). In one embodiment, the misoprostol and the antibiotic are comprised in an extended release dosage form. In one embodiment, the misoprostol and/or the antibiotic is released directly to the colon.

In some embodiments, the dose of misoprostol comprises a range from 10 μg to 1000 μg. In some embodiments, the dose of misoprostol comprises a range from 50 μg to 500 μg. In some embodiments, the dose of misoprostol comprises a range from 100 μg to 200 μg. In one embodiment, the dose of misoprostol is about 10 μg, about 25 μg, about 50 μg, about 75 μg, about 100 μg, about 125 μg, about 150 μg, about 175 μg, about 200 μg, about 250 μg, about 300 μg, about 350 g, about 400 μg, about 450 μg, about 500 μg, about 600 μg, about 700 μg, about 800 μg, about 900 μg, about 1000 μg, or more.

In some embodiments, the dose of vancomycin (or another antibiotic disclosed herein) comprises a range from 10 mg to 1000 μg. In some embodiments, the dose of misoprostol comprises a range from 50 mg to 500 mg. In some embodiments, the dose of misoprostol comprises a range from 100 mg to 200 mg. In one embodiment, the dose of misoprostol is about 10 mg, about 25 mg, about 50 mg, about 75 mg, about 100 mg, about 125 mg, about 150 mg, about 175 mg, about 200 mg, about 250 mg, about 300 mg, about 350 mg, about 400 mg, about 450 mg, about 500 mg, about 600 mg, about 700 mg, about 800 mg, about 900 mg, about 1000 mg, or more.

In some embodiments, the active compound (for example, misoprostol) is taken 1, 2, 3, 4, 5, 6, 7, 8, or more times daily. In some embodiments, the antibiotic (for example, vancomycin) is taken 1, 2, 3, 4, 5, 6, 7, 8, or more times daily.

In some embodiments, the active compound (for example, misoprostol) is taken for about 1 days, about 2 days, about 3 days, about 4 days, about 5 days, about 6 days, about 7 days, about 8 days, about 9 days, about 10 days, about 14 days, about 21 days, about 28 days, or more days. In some embodiments, the antibiotic (for example, vancomycin) is taken for about 1 days, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, or more days.

In some embodiments, the misoprostol is administered two times daily for 14 days, wherein each dose of misoprostol is 200 μg and the vancomycin is administered four times daily for 10 to 14 days, wherein each dose of vancomycin is 125 mg. In some embodiments, the misoprostol is administered two times daily for 14 days, wherein each dose of misoprostol is 200 μg and the vancomycin is administered four times daily for 10 to 14 days, wherein each dose of vancomycin is 125 mg, and wherein the misoprostol is administered on the final four days of vancomycin treatment.

In some embodiments, the misoprostol is administered 4 times daily for 14 days, wherein each dose of misoprostol is 200 μg and the vancomycin is administered 4 times daily for 10 to 14 days, wherein each dose of vancomycin is 125 mg.

In one aspect, provided herein is a method for treating or preventing the recurrence of a C. difficile infection comprising administering to a host in need thereof an effective amount of misoprostol, or a pharmaceutically acceptable salt thereof, and an additional therapeutic agent.

In one embodiment, the additional therapeutic agent is selected from the group consisting of small molecules, monoclonal antibodies, toxoid vaccines, subunit vaccines, recombinant enzymes, biologics, peptides, recombinant proteins, oligonucleotides, and polymers.

In one embodiment, the additional therapeutic agent is a small molecule. Small molecules include, for example, fidaxomicin, metronidazole, teicoplanin, vancomycin, cadazolid, DAV-132, OPS-2071, ramoplanin, ridinilazole, CRS-3123, DS-2969, MCB-3681, MCB-3837, MGBBP-03, miconazole nitrate, ATx-401, INS-5010, MBX-500, OG-253, SQ-641, VAL-301, INS-001, INS-201, AQP-182, GBV-003, GBV-006, NPI-32101, NVB-302, RBx-14255, rifalazil, SMT-21829, SQ-109, LFF-571, surotomycin, and/or P-100031. In some embodiments, the additional therapeutic agent is an antibiotic.

In one embodiment, the additional therapeutic agent is a monoclonal antibody. Monoclonal antibodies include, for example, bezlotoxumab, PolyCAb, antibodies for enterocolitis, EV-209104, EV-029105A, IMM-529, OraCAb, PRO-391, PA-41, and/or PA-50. In one embodiment, the additional therapeutic agent is bezlotoxumab (Zinplava). In some embodiments, the misoprostol is administered in combination with bezlotoxumab (Zinplava). In some embodiments, the misoprostol is administered in combination with an antibiotic and bezlotoxumab (Zinplava). The bezlotoxumab can be given as a single dose (Wilcox M H, et. al. Bezlotoxumab for Prevention of Recurrent Clostridium difficile Infection. N Engl J Med. 2017 Jan. 26; 376(4):305-317).

In one embodiment, the additional therapeutic agent is a toxoid vaccine. Toxoid vaccines include, for example, PF-06425090.

In one embodiment, the additional therapeutic agent is a subunit vaccine. Subunit vaccines include, for example, VLA-84.

In one embodiment, the additional therapeutic agent is a recombinant enzyme.

Recombinant enzymes include, for example, ribaxamase, SYN-006, SYN-007, P-4A, and/or P-1-A.

In one embodiment, the additional therapeutic agent is a biologic. Biologics include, for example, RBX-2660, SER-109, SER-262, ABM-101, ampliphage-004, RBX-7455, SHP-01, CBM-588, endolysin, and/or VP-20621.

In one embodiment, the additional therapeutic agent is a peptide. Peptides include, for example, AP-114, CD-17DL, and/or NP-432.

In one embodiment, the additional therapeutic agent is a recombinant protein. Recombinant proteins include, for example, AvR2-V 10.

In one embodiment, the additional therapeutic agent is an oligonucleotide. Oligonucleotides include, for example, Cdiff snare. An oligonucleotide can also include an RNA/DNA vaccine that leads to in vivo expression of an immunogen or biologic therapy.

In one embodiment, the additional therapeutic agent is a polymer. Polymers include, for example, tolevamer.

Combination Therapy with Probiotics

Probiotics are a class of microorganisms defined as live microbial organisms that beneficially affect the animal and human hosts. The beneficial effects include improvement of the microbial balance of the intestinal microflora or improving the properties of the indigenous microflora. The beneficial effects of probiotics may be mediated by a direct antagonistic effect against specific groups of organisms, resulting in a decrease in numbers, by an effect on their metabolism or by stimulation of immunity. The mechanisms underlying the proposed actions remain vastly unknown, partly as a consequence of the complexity of the gastro-intestinal ecosystem with which these biotherapeutic agents are expected to interact. Probiotics may suppress viable counts of an undesired organism by producing antibacterial compounds, by competing for nutrients or for adhesion sites. Further, they may alter microbial metabolism by increasing or decreasing enzyme activity or they may stimulate the immune system by increasing antibody levels or increasing macrophage activity. Probiotics may have antimicrobial, immunomodulatory, anti-carcinogenic, anti-diarrheal, anti-allergenic and antioxidant activities. Known probiotic strains include, for example, Bifidobacteria, Lactobacillus, Lactococcus, Saccharomyces, Streptococcus thermophilus, Enterococcus and E. coli.

Non-limiting examples of probiotics for use in combination with misoprostol for the treatment or prevention of C. difficile infections include, but are not limited to species of: Lactobacillus, Bifidobacterium, Non-difficile Clostridium, Non-toxigenic Clostridium difficile, Saccharomyces, or Streptococcus. (See Ollech J E, Shen N T, Crawford C V, Ringel Y. Use of probiotics in prevention and treatment of patients with Clostridium difficile infection. Best Practice & Research Clinical Gastroenterology. 2016; 30(1):111-8).

In one embodiment, disclosed herein are compositions comprising misoprostol, or a pharmaceutically acceptable salt thereof, and a probiotic.

In one embodiment, disclosed herein is a method for treating or preventing the recurrence of a C. difficile infection comprising administering to a host in need thereof an effective amount of misoprostol, or a pharmaceutically acceptable salt thereof, and a probiotic.

In one embodiment, the probiotic is selected from the group consisting of Lactobacillus species, Bacillus species, Bifidobacterium species, Non-difficile clostridia, Non-toxigenic Clostridium difficile, Saccharomyces species, and Streptococcus species.

In one embodiment, the probiotic is Lactobacillus species. In one embodiment, the probiotic is Bacillus species. In one embodiment, the probiotic is Bifidobacterium species. In one embodiment, the probiotic is Non-difficile Clostridium species. In one embodiment, the probiotic is Non-toxigenic Clostridium difficile. In one embodiment, the probiotic is Saccharomyces. In one embodiment, the probiotic is Streptococcus.

In one embodiment, the probiotic is administered prior to the misoprostol. In one embodiment, the probiotic is administered concurrently with the misoprostol. In one embodiment, the probiotic is administered after the misoprostol.

In some embodiments, the misoprostol and the probiotic are combined in a single dosage form (for example, in a single oral tablet). In one embodiment, the misoprostol and the probiotic are comprised in an extended release dosage form. In one embodiment, the misoprostol and/or the probiotic are released directly to the colon.

In some embodiments, the dose of misoprostol comprises a range from 10 μg to 1000 μg. In some embodiments, the dose of misoprostol comprises a range from 50 μg to 500 μg. In some embodiments, the dose of misoprostol comprises a range from 100 μg to 200 μg. In one embodiment, the dose of misoprostol is about 10 μg, about 25 μg, about 50 μg, about 75 μg, about 100 μg, about 125 μg, about 150 μg, about 175 μg, about 200 μg, about 250 μg, about 300 μg, about 350 μg, about 400 μg, about 450 μg, about 500 μg, about 600 μg, about 700 μg, about 800 μg, about 900 μg, about 1000 μg, or more.

In some embodiments, the active compound (for example, misoprostol) is taken 1, 2, 3, 4, 5, 6, 7, 8, or more times daily. In some embodiments, the probiotic is taken 1, 2, 3, 4, 5, 6, 7, 8, or more times daily.

In some embodiments, the active compound (for example, misoprostol) is taken for about 1 days, about 2 days, about 3 days, about 4 days, about 5 days, about 6 days, about 7 days, about 8 days, about 9 days, about 10 days, about 14 days, about 21 days, about 28 days, or more days. In some embodiments, the probiotic is taken for about 1 days, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, or more days.

### Compositions

Compositions, as described herein, comprising an active compound and an excipient of some sort may be useful in a variety of applications.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005). The pharmaceutically acceptable excipients may also include one or more of fillers, binders, lubricants, glidants, disintegrants, and the like.

Exemplary excipients include, but are not limited to, any non-toxic, inert solid, semi-solid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), bucally, or as an oral or nasal spray.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, varoius gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacilic acid/acrylamide copolymer, polyalkylene oxides such as polyethylene oxide, polypropylene oxide, poly(ethylene oxide-propylene oxide), and a Pluronic polymer, polyoxyethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly(meth)acrylic acid, and esters amide and hydroxyalkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

In some embodiments, the misoprostol is delivered directly to the colon. The direct delivery to the colon may avoid some of the side effects of the misoprostol.

The dosage form may include a coating. The tablet may be coated with one or more enteric polymers or pharmaceutically acceptable seal coat polymers.

In some embodiments, misoprostol and an antibiotic (or a probiotic) may be administered in combination with diclofenac sodium. Arthrotec® is an example of a combination product containing diclofenac sodium, a non-steroidal anti-inflammatory drug (NSAID) with analgesic properties, and misoprostol, a gastrointestinal (GI) mucosal protective prostaglandin E1 analog. Each Arthrotec tablet consists of an enteric-coated core containing 50 mg or 75 mg diclofenac sodium surrounded by an outer mantle containing 200 μg misoprostol.

### Extended Release Compositions

In one aspect of the present invention, an effective amount of an active compound (for example misoprostol; misoprostol and an antibiotic; misoprostol and a probiotic) as described herein is incorporated into nanoparticles, e.g. for convenience of delivery and/or extended release delivery. The use of materials in nanoscale provides one the ability to modify fundamental physical properties such as solubility, diffusivity, blood circulation half-life, drug release characteristics, and immunogenicity. In the last two decades, a number of nanoparticle-based therapeutic and diagnostic agents have been developed for the treatment of cancer, diabetes, pain, asthma, allergy, and infections. These nanoscale agents can provide more effective and/or more convenient routes of administration, lower therapeutic toxicity, extend the product life cycle, and ultimately reduce health-care costs. As therapeutic delivery systems, nanoparticles allow targeted delivery and controlled release. Nanoparticles can also be used to allow targeted delivery and controlled release of oligonucleotide therapies and oligonucleotide compositions.

In addition, nanoparticle-based drug delivery can be used to release drugs at a sustained rate and thus lower the frequency of administration, deliver drugs in. a target manner to minimize systemic side effects, or deliver two or more drugs simultaneously for combination therapy to generate a synergistic effect and suppress drug resistance. To date, a number of nanotechnology-based therapeutic products have been approved for clinical use. Among these products, liposomal drugs and polymer-based conjugates account for more than 80% of the products. See, Zhang, L., et al., Nanoparticles in Medicine: Therapeutic Applications and Developments, Clin. Pharm. and Ther., 83(5):761-769, 2008.

Nanoparticles may be prepared using a wide variety of methods known in the art. For example, nanoparticles can be formed by methods as nanoprecipitation, flow focusing fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci, 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; U.S. Pat. Nos. 5,578,325 and 6,007,845; P. Paolicelli et al., “Surface-modified PLGA-based Nanoparticles that can Efficiently Associate and Deliver Virus-like Particles” Nanomedicine. 5(6):843-853 (2010)).

In some embodiments, the compounds described herein are associated with a nanoparticle, such as a polymeric nanoparticle. Nanoparticles may comprise natural polymers, including but not limited to chitosan, alginate, dextran, gelatin, and albumin, and synthetic polymers such as, but not limited to, poly(lactide-co-glycolide) (PLGA), (3-hydroxybutyrate-co-3-hydroxyvalerate) (PHBV), poly(sebacic anhydride), poly(e-caprolactone), polystyrene, thermoresponsive (i.e., NIPAAm and CMCTS-g-PDEA) and pH-responsive (i.e., Eudragit LI 00, Eudragit S and AQOAT AS-MG) polymers.

In one embodiment, the polymeric particle is between about 0.1 nm to about 10000 nm, between about 1 nm to about 1000 nm, between about 10 nm and 1000 nm, between about 100 nm and 800 nm, between about 400 nm and 600 nm, or about 500 nm. In one embodiment, the micro-particles are about 0.1 nm, 0.5 nm, 1.0 nm, 5.0 nm, 10 nm, 25 nm, 50 nm, 75 nm, 100 nm, 150 nm, 200 nm, 250 nm, 300 nm, 400 nm, 450 nm, 500 nm, 550 nm, 600 nm, 650 nm, 700 nm, 750 nm, 800 nm, 850 nm, 900 nm, 950 nm, 1000 nm, 1250 nm, 1500 nm, 1750 nm, or 2000 nm.

In some embodiments, the misoprostol and the antibiotic are combined in a single dosage form (for example, in a single oral tablet). In one embodiment, the misoprostol and the antibiotic are comprised in an extended release dosage form.

In one embodiment, the tablet dosage form comprises:
an inner tablet comprising misoprostol or a salt thereof and optionally other pharmaceutically acceptable excipients; and
an outer tablet comprising an antibiotic or a salt thereof and optionally other pharmaceutically acceptable excipients.

In one embodiment, the tablet dosage form comprises:
an inner tablet comprising an antibiotic or a salt thereof and optionally other pharmaceutically acceptable excipients; and
an outer tablet comprising misoprostol or a salt thereof and optionally other pharmaceutically acceptable excipients.

In some embodiments, the misoprostol and the probiotic are combined in a single dosage form (for example, in a single oral tablet). In one embodiment, the misoprostol and the probiotic are comprised in an extended release dosage form.

In one embodiment, the tablet dosage form comprises:
an inner tablet comprising misoprostol or a salt thereof and optionally other pharmaceutically acceptable excipients; and
an outer tablet comprising a probiotic or a salt thereof and optionally other pharmaceutically acceptable excipients.

In one embodiment, the tablet dosage form comprises:
an inner tablet comprising a probiotic or a salt thereof and optionally other pharmaceutically acceptable excipients; and
an outer tablet comprising misoprostol or a salt thereof and optionally other pharmaceutically acceptable excipients.

### EXAMPLES

The following examples are set forth below to illustrate the systems, methods, compositions and results according to the disclosed subject matter. These examples are not intended to be inclusive of all aspects of the subject matter disclosed herein, but rather to illustrate representative systems, methods, compositions and results. These examples are not intended to exclude equivalents and variations of the present invention which are apparent to one skilled in the art.

### Example 1. Misoprostol for Treatment and Prevention of Clostridium difficile Colitis

Clostridium difficile infection (CDI) is a leading nosocomial infection and the primary identifiable cause of antibiotic-associated diarrhea. It can lead to multiple CDI recurrences, sepsis, toxic megacolon, and death. Recurrent CDI complicates 20% of primary episodes of disease and once someone has had a recurrence, the risk for subsequent episodes increases dramatically. Recurrent CDI is associated with an increased risk of death and a poor quality of life. While manipulation of the gut microbiome through probiotics or fecal transplantation has met with some success in breaking the cycle of recurrent CDI, there remains a need for simple, safe, and cost-effective solutions to this problem.

Prostaglandins (PGs) are endogenous lipid mediators generated by the cyclooxygenase-dependent metabolism of arachidonic acid. They are involved in many aspects of health and disease. It is for the latter reason that people take nonsteroidal anti-inflammatory drugs (NSAIDs), which inhibit PG synthesis and alleviate pain, fever, and inflammation. Because PGs, especially PGE2, are important to gut health, NSAID use can lead to gastrointestinal problems such as stomach ulcers. In fact, misoprostol, a PGE analogue that binds to all four of PGE2's EP receptors, is FDA approved to prevent ulcers in patients taking NSAIDs. A phenome-wide association study (PheWAS) was performed based on ICD-9 billing code and genotype data in a disease-agnostic cohort of ˜40,000 patients to identify potential novel genotype-phenotype associations related to the SNPs in the genes for the targets of misoprostol (PGE2 receptors). This PheWAS analysis determined a potential new indication for misoprostol to treat (or prevent) C. difficile colitis. PheWAS can be thought of as a “reverse GWAS”—determining, for a given genotype, the range of associated clinical phenotypes (see Innovation for details).

CDI is the most commonly diagnosed infectious etiology of antibiotic-associated diarrhea and has surpassed methicillin resistant Staphylococcus aureus as the most common healthcare associated infections in many US hospitals (4). Nearly 14,000 people die each year in the US from CDI. The attributable mortality of CDI is 5-10% but is disproportionately lethal in the elderly. A major challenge of CDI is recurrence, which can impact 20-30% of patients and is associated with an increased risk of death. Treatment refractory, recurrent CDI has resulted in the use of fecal microbiota transplants (FMT) for curing or inducing remission. However, problems with standardization, availability, and putative risks from FMT have made this form of therapy suboptimal.

As they prevent synthesis of endogenous prostaglandins (PGs), nonsteroidal anti-inflammatory drugs (NSAIDs) can adversely affect gut health. Epidemiological studies reveal an association between CDI risk and the use of NSAIDs (Dial S, et al. JAMA. 2005; 294(23):2989-95; Suissa D, et al. Br J Clin Pharmacol. 2012; 74(2):370-5; Soes L M, et al. Epidemiol Infect. 2014; 142(7):1437-48), underscored by a recent meta-analysis (Permpalung N, et al. Association between nonsteroidal anti-inflammatory drugs and Clostridium difficile-associated diarrhea: A systematic review and meta-analysis. Can J Gastroenterol Hepatol. 2015). Apropos to CDI, older adults are major consumers of NSAIDs. The plausibility of a link between NSAID use and CDI is bolstered by the association between NSAID use and flare-ups of inflammatory bowel disease (Kvasnovsky C L, et al. Scandinavian journal of gastroenterology. 2014:1-9) and the occasional occurrence of NSAID-associated colitis (Tonolini M. Journal of emergencies, trauma, and shock. 2013; 6(4):301-3. PMCID: Pmc3841543; Riddell R H, et al. Gut. 1992; 33(5):683-6; Gentric A, Pennec Y L. Lancet. 1992; 340(8811):126-7; Romero-Gomez M, et al. Journal of clinical gastroenterology. 1998; 26(3):228).

Animal and human studies suggest that CDI provokes local and systemic increases in PGE2 (Lauritsen K, et al. Gastroenterology. 1988; 95(1): 11-7; Kim H, et al. J Biol Chem. 2005; 280(22):21237-45; Triadafilopoulos G, et al. Gastroenterology. 1989; 97(5):1186-92; Stratton Md., et al. Prostaglandins. 1994; 48(6):367-75; Czepiel J, et al. J Physiol Pharmacol. 2014; 65(5):695-703; Alcantara C, et al. The Journal of infectious diseases. 2001; 184(5):648-52).

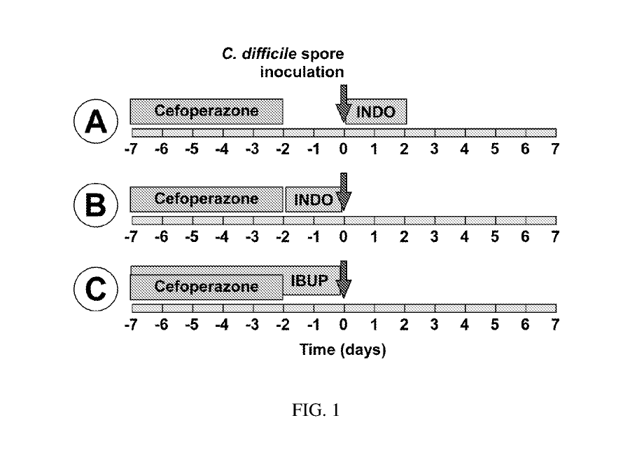

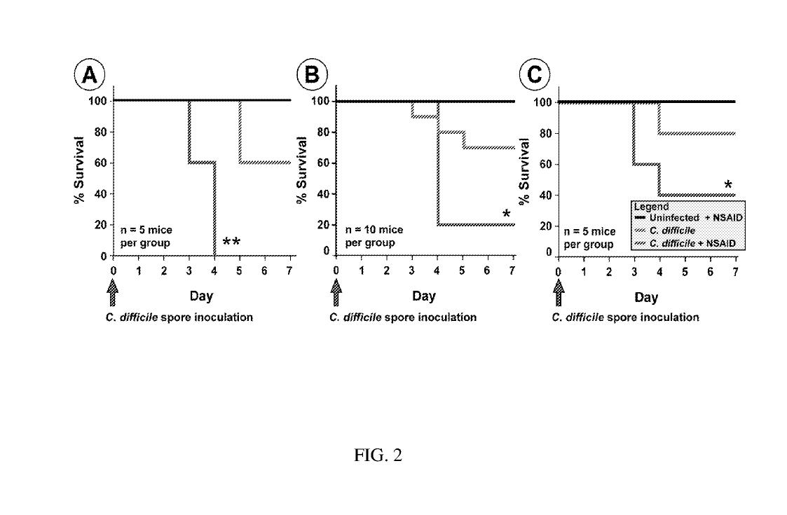

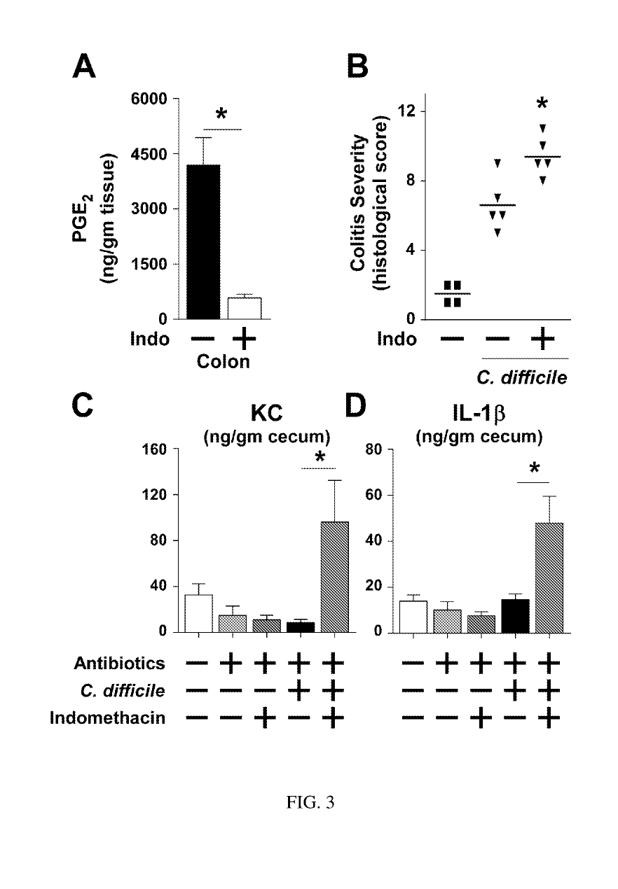

The extent to which this release of PGE2 modulates disease is unclear. As shown in FIGS. 1 and 2, treatment of mice with the cyclooxygenase inhibitor indomethacin or ibuprofen before or after infection resulted in higher mortality in CDI caused by a clinical NAP1/027 isolate (and another strain of C. difficile, 630, data not shown). It was confirmed that indomethacin suppressed tissue PGE2 levels and induced more tissue damage and acute inflammation (FIG. 3).

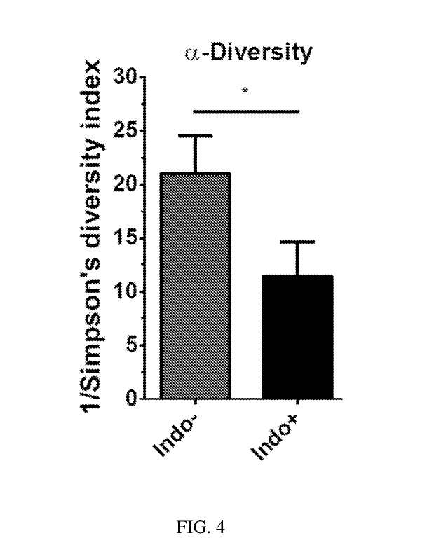

A possible mechanism for how PGE2 modulates disease could be through the microbiome. NSAIDs have been implicated as potentially disrupting the gut microbiome (Syer S D, et al. J Gastroenterol. 2015; 50(4):387-93; Blackler R W, et al. American journal of physiology Gastrointestinal and liver physiology 2015; 308(12):G994-1003; Tiihonen K, et al. The British journal of nutrition. 2008; 100(1):130-7; Teran-Ventura E, et al. Journal of Crohn's & colitis. 2014; 8(9):1043-54; Makivuokko H, et al. The British journal of nutrition. 2010; 103(2):227-34; Aronoff D M, Rogers M A M. The Influence of Nonsteroidal Anti-Inflammatory Drugs on the Gut Microbiome. Clinical Microbiology and Infection. 2015; In press). It was found that exposing mice to indomethacin for 2 days significantly altered microbial diversity in the colon (FIG. 4).

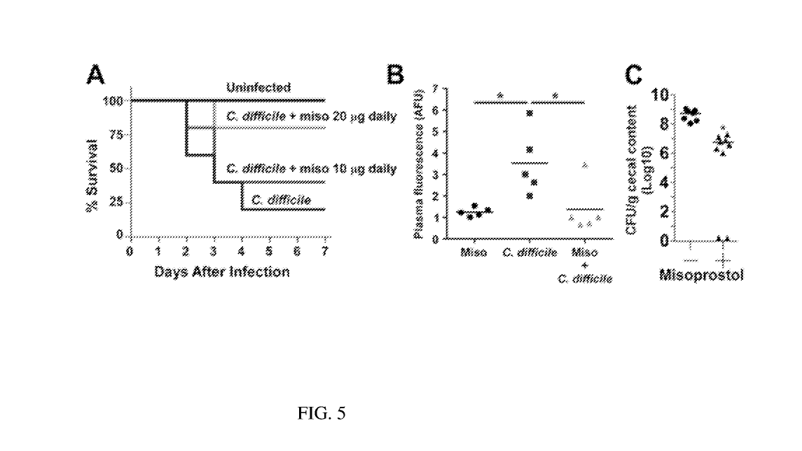

The next experiment was whether exogenous PGE2 receptor stimulation with misoprostol, a stable PGE1 analogue that binds all 4 of the PGE2 EP receptors (Kiriyama M, et al. Br J Pharmacol. 1997; 122(2):217-24) could improve outcome in an acute CDI mouse model. As shown (FIGS. 5A & B), misoprostol reduced CDI severity. It prevented death and loss of gut integrity in mice infected with the NAP1 strain M7404. It also reduced C. difficile colonization levels (FIG. 5C) and reduced diarrhea (not shown).

Misoprostol (Cytotec) is an FDA-approved drug that provides gastric protection against NSAID-associated ulcers by increasing the cytoprotective levels of PGE2 necessary for maintaining integrity of the gastric mucosa (Fashner J, Gitu A C. American family physician. 2015; 91(4):236-42). It is also used off-label for a variety of indications in obstetrics and gynecology, including medication abortion, induction of labor, and treatment of postpartum hemorrhage. Misoprostol has a long safety record and has been extensively studied and used in humans and rodents. The idea that prostaglandin signaling can be targeted as an approach to prevent CDI is new and innovative.

Dose studies with misoprostol Mice are treated with misoprostol (delivered by oral gavage daily) immediately following the termination of vancomycin therapy. As previously published (Lawson J A, et al. Australian and New Zealand Journal of Surgery. 1994; 64(3):197-201), disease severity slowly returns to baseline in C. difficile-infected mice not treated with vancomycin and relapse does not occur. While vancomycin treatment accelerates resolution of colitis it results in a gradual return of colitis by day 16, with increased colonization levels and C. difficile toxin production (data found in Lawson J A, et al. Australian and New Zealand Journal of Surgery. 1994; 64(3):197-201).

In one example, misoprostol is given daily by oral delivery. A Bayesian adaptive study design in mice and start with a dose of misoprostol that was effective in treating acute CDI. The primary outcome in these experiments is histopathological severity of colitis, scored blindly by a trained histopathologist to prevent bias (Trindade B C, et al. Anaerobe. 2014; 30C:90-8; Lawson J A, et al. Australian and New Zealand Journal of Surgery. 1994; 64(3):197-201). Secondary outcomes, which serve as important surrogates for disease activity, include weight loss (measured daily), sickness behavior (assessed daily via modified SHIRPA score (Collins J, et al. Microbiome. 2015; 3:35)) the colonization burden of C. difficile per gram of stool (measured daily), diarrhea severity (scored daily (Sun X, et al. Infect Immun. 2011; 79(7):2856-64)), and the amount of toxin shed in the stool (measured on days 0, 4, 9, and 16) (Theriot C M, et al. Gut Microbes. 2011; 2(6):326-34. PMCID: 3337121; Lawson J A, et al. Australian and New Zealand Journal of Surgery. 1994; 64(3): 197-201). Apart from SHIRPA scores, each of these outcomes are quantitative measurements, which reduces bias. Toxin and bacterial burden in the stool are measured by someone blinded to treatment conditions.

Timing Studies with Misoprostol

The next investigation determines the best time to dose misoprostol to prevent relapse: following treatment, during and after treatment, or just during treatment. Mice are given daily misoprostol starting at the conclusion of antibiotic treatment for CDI (vancomycin). Relapse is anticipated to occur between days 9 and 16 following cessation of vancomycin. In another example, misoprostol is administered starting during the treatment phase of CDI (concomitant with vancomycin) and continued through the period of expected relapse. In yet another example, misoprostol is administered only during treatment with vancomycin. For select experiments, cohorts of mice are observed for two additional weeks following cessation of misoprostol (to day +30) for evidence of delayed relapse.

Misoprostol can also be used in conjunction with antibiotics, or with additional therapeutics (such as a monoclonal antibody), to treat acute CDI. The data in this example already show that misoprostol alone can treat CDI. Misoprostol could also reduce the dose or duration of anti-difficile antibiotics (like vancomycin) anti-difficile antibodies (such as bezlotoxumab) and can help support recovery of a healthy gut microbiome more rapidly.

### Example 2. Efficacy and Safety of Misoprostol and Vancomycin Versus Vancomycin in Elderly Patients with First Recurrence of Clostridium Difficile Infection

### Introduction and Background

Clostridium difficile is a Gram-positive, spore-forming, toxin-producing bacillus that causes an increasing amount of infectious diarrhea and colitis. Clinical recurrence of infection occurs in about 25% of patients initially treated with antibiotic therapy. Antibiotic treatment of patients with clinical recurrence of infection often still prevent further recurrence, particularly in the elderly, defined as age 65 and over. Unusual measures with some risk such as fecal transplant are being investigated for the therapy of patients with clinically recurrent infection. Misoprostol, a PGE1 analogue, has been found to enhance recovery from Clostridium difficile infection in animal models, and a deficient PGE1 pathway is associated with colitis in a PheWAS analysis. Misoprostol is approved by the FDA for chronic use in the prevention of gastric ulcers, among other indications. This Example evaluates the rate of clinically recurrent infection in patients treated with misoprostol and vancomycin, compared to vancomycin alone in elderly patients with a first recurrence of C. difficile infection.

The efficacy and safety objectives for this study are as follows: 1) Determine if misoprostol can modify the rate of clinical recurrence of C. difficile infection in elderly patients during the first 14, 28, and 90 days after vancomycin therapy is stopped, and 2) Determine the safety of misoprostol in elderly patients with recurrent C. difficile infection compared to placebo as measured by treatment emergent adverse events and standard laboratory assessments.

### Methods

This is a multicenter, randomized, double-blind, placebo-controlled, study in elderly patients with first recurrence of Clostridium difficile infection CDI). Patient enrollment is based on the presence of diarrhea (three unformed stools or more in 24 hours before randomization), and the presence of C. difficile toxin A or B in stool samples within 48 hours of randomization. Patients will have had a single prior CDI episode within the past 3 months. Concomitant treatment with other potentially effective treatments for CDI is not allowed. All enrolled patients take standard oral vancomycin 125 mg four times a day for 10 to 14 days, and investigational oral misoprostol or placebo four times a day for 14 days. Dosing of misoprostol or placebo starts within 48 hours of clinical resolution of diarrhea, and resolution of serious CDI infection signs and symptoms (resolution on average day 3 on vancomycin therapy). Patients are stratified based upon their clinical presentation of mild to moderate versus severe CDI. 1:1 randomization of misoprostol and placebo, within the stratified groups.

A total of 80 patients are enrolled in this study. It is anticipated that one third to one half of patients treated with vancomycin and placebo will have a second recurrence of CDI within 28 days of completing the vancomycin therapy.

Patients randomized to misoprostol take four 200 μg tablets by mouth per day (800 μg daily dose, four daily doses) for 14 days. Patients randomized to placebo take four matching placebo capsules by mouth per day for 14 days.

### Inclusion Criteria

1. Adult male or female patients >65 years of age with CDI, and a single past CDI within the past 3 months (Alternatively, >50 years of age may also be used).
2. Initial response to vancomycin therapy with clinical resolution of diarrhea, and resolution of serious CDI signs and symptoms.
3. Both outpatient and inpatient groups are eligible.

### Exclusion Criteria

1. Be less than 65 years of age. (Alternatively, less than 50 years of age may be used for exclusion)
2. Pregnant, nursing, or planning to become pregnant.
3. Current or planned treatment with prostanoid therapy.
4. Significant lung (FVC<70%), liver (cirrhosis), or kidney (GFR<60) disease.
5. Known hypersensitivity to vancomycin or misoprostol.
6. Recent myocardial infarction.
7. Have taken investigational drugs within 30 days before Investigational Medicinal Product
   (IMP) administration.
8. Inability to understand the requirements of the study, inability to abide by the study restrictions and to return for the required treatments and assessments.
9. Otherwise unsuitable for the study, in the opinion of the investigator.

### Endpoints

To evaluate the exploratory objectives of the study assessing oral misoprostol compared to placebo in elderly patients with a first recurrence of CDI, the following endpoints are measured:
Determine if misoprostol can modify the rate of clinical recurrence of C. difficile infection in elderly patients during the first 14, 28, and 90 days after vancomycin therapy is stopped
Determine the safety of misoprostol in elderly patients with recurrent C. difficile infection compared to placebo as measured by treatment emergent adverse events and standard laboratory assessments.
Resolution of toxin A and B presence.
Assess clinician concern with overlap of symptoms between adverse events associated with misoprostol, and worsening of CDI

Assessments by interview, recording of adverse events, physical examination, clinical laboratory tests, and other evaluations.

Patients are asked to record self-administration of study drugs and CDI symptoms.

Assessments may be collected at additional time-points based upon regularly scheduled visits.

### Example 3. A Randomized, Double-Blind, Placebo-Controlled Trial to Assess the Efficacy and Safety of Misoprostol in the Prevention of First Recurrence of Clostridium difficile Infection in Adults Aged 50 and Over

The study described in this example is used to: evaluate the safety and tolerability of misoprostol dosed orally for up to 14 days in older adults (50 years of age) being treated with the standard of care for C. difficile infection (CDI), measured by treatment emergent adverse events and standard laboratory assessments; evaluate the efficacy of misoprostol for prevention of recurrent CDI (rCDI), by determining if misoprostol can modify the rate and severity of rCDI in adults 50 years of age during the first 8 weeks after standard oral vancomycin therapy is completed; and determine the effect of misoprostol on the gut microbiome and diarrhea occurrence/severity.

### Introduction

Clostridium difficile is a Gram-positive, spore-forming, toxin-producing bacillus that causes diarrhea and colitis often following exposure to antibiotics. It is emerging as one of the most common healthcare-associated infections. C. difficile infection (CDI) ranges in severity from mild diarrhea to severe colitis (sometimes resulting in septic shock and/or death). In addition, CDI has a high tendency to recur following treatment. First clinical recurrence of infection occurs in about 25% of patients initially treated with antibiotic therapy (rate of recurrence can be much higher among those 50 years of age). Once a first recurrence has occurred, the risk for future recurrences exceeds 40% (again, higher in older adults, 50 years of age). The problem of recurrent CDI (rCDI) is a significant one, negatively impacting quality of life. There are patients for whom the cycle of recurrences does not remit. Experimental treatments such as fecal transplant are being investigated for patients with rCDI. Misoprostol, a prostaglandin analogue, has been shown herein to enhance recovery from CDI in animal models, and decreased prostaglandin pathway signaling is associated generally with GI inflammatory conditions in a phenome wide association study (PheWAS) analysis. Misoprostol is FDA-approved for chronic use in the prevention of gastric ulcers given its established mucosal protective properties and inhibitory effects on gastric acid. This trial is used to evaluate the rate and severity of rCDI in patients treated with oral misoprostol compared to placebo subsequent to the initiation of standard of care (SOC) front line treatment with oral vancomycin in adults aged 50 years or older following a primary episode of CDI.

### Background

Clostridium difficile

CDI is a serious problem worldwide, and in the United States, it comprises approximately 17.1% of hospital-associated infections affecting 500,000 individuals annually with an incidence that is climbing yearly. It leads to worse clinical outcomes, more frequent readmissions, longer hospital stays, and costs billions of dollars each year in health care expenses. In this disease, there are focal areas of epithelial loss and an exudate consisting of polymorphonuclear cells, fibrin, and cellular debris. Focal inflammation might arise after exposure to high local concentrations of toxins released from CDI adherent to specific small regions of the epithelium. There are two main toxins that cause damage, Toxins A and B, and these cause the symptoms of CDI.

The most well-known risk factor for the infection is antibiotic use, but other risk factors include advanced age, hospitalization within preceding 2 months, proton-pump inhibitor use, and severity of underlying illness. Inflammatory bowel disease (IBD), which includes Crohn's disease (CD), ulcerative colitis (UC), and patients after ileal pouch anal anastomosis (IPAA), is known to be an independent risk factor for CDI (Hughes M, et al. Inflamm Bowel Dis. 2016 April; 22(4):853-861). A history of CDI has also been associated with failure of IPAA reconstruction, which is a frequent treatment for UC (Skowron K B, et al. Inflamm Bowel Dis. 2016 April; 22(4):902-911).

Humoral immunity is an increasing area of interest in understanding the varied clinical presentation of CDI in both IBD and non-IBD patients, and also in investigating why an IBD patient may be more susceptible to clinical disease. Hughes et al. (2016) conducted a study to determine if IBD patients have any alteration in humoral response to CDI toxins A or B when compared with a group of control patients enrolled from outpatient clinics at the same medical center. They found that patients with UC have lower IgA levels to CDI toxins compared to those with Crohn's disease and those after IPAA. Patients with IBO with prior CDI failed to demonstrate any increase in antitoxin IgG. These findings suggest that IBD patients may benefit from immunization strategies targeting CDI toxins. However, if prostaglandins maintain mucosal integrity and enhance almost all mucosal defensive mechanisms, perhaps they can protect against other exogenous insults, lower in the GI system.

A prostaglandin E2 receptor 4 (EP4) receptor agonist, KAG-308, is in Phase II study for ulcerative colitis [KAKEN]. Oral administration of KAG-308 suppressed onset of dextran sulfate sodium-induced colitis and promoted mucosal healing in a mouse model; it also prevented colorectal carcinogenesis by inhibiting colitis development in another mouse model (conversely, an EP4 antagonist increased mortality) (Watanabe Y, et al. Eur J Pharmacol. 2015 May 5; 754:179-189. PMID: 25704618).

### Phe WAS in Drug Discovery and Drug Repurposing

One source of data guiding this study is BioVU, a large repository of de-identified DNA samples that Vanderbilt University Medical Center has catalogued for >10 years from what otherwise would have been excess, discarded patient blood samples collected during routine clinical testing. This biobank is a centralized resource for investigating genotype-phenotype associations. Biospecimens within BioVU are linked to corresponding longitudinal clinical and demographic data derived from the Synthetic Derivative, a de-identified database of EMRs (Roden D M, et al. Clin Pharmacol Ther. 2008 September; 84(3):362-369. PMCID: PMC3763939; Bowton E, et al. Sci Transl Med. 2014 April; 6(234):234). More recently, PheWAS has been introduced as a systematic and efficient approach to discover novel disease-variant associations and pleiotropy using BioVU. It is the comprehensive and diverse nature of the diagnostic information within EMRs that enables PheWAS. PheWAS not only replicates known genetic-phenotypic associations, but also reveals new phenotypic associations with genetic variants, enhancing analyses of the genomic basis of human diseases and providing genetic support for drug discovery and drug repurposing efforts (Pulley J M, et al. ASSAY Drug Dev Technol. 2017 April; 15(3): 113-119).

Prostaglandin receptor function and misoprostol pharmacology Prostaglandin E2 (PGE2) plays a pivotal role in maintaining local homeostasis in a variety of pathophysiological settings, including the colon. PGE2 receptors (EPs) mediate the effects of this molecule and include four subtypes: EP1-4. PGE2 participates decisively in the defense of the colonic mucosa. For example, misoprostol, a synthetic PGE1 analog, potently protects human colonic mucosa against mucosal insults. In addition, in mice, PGE2 and EP4-selective agonists significantly improved colitis induced by dextran sodium sulfate (DSS) treatment. Nakatsuji et al. (2015) investigated the effect of the recently identified EP4-association protein (EPRAP), which is essential for anti-inflammatory function of EP4 signaling in macrophages, on colitis and colitis-associated tumorigenesis in mice. They observed that EPRAP deficiency exacerbated DSS-induced colitis and found support for the idea that EPRAP in macrophages functions crucially in suppressing colonic inflammation (Nakatsuji M, et al. PLOS Genet. 2015 Oct. 6; 11(10):e1005542).

Misoprostol is utilized clinically as an anti-ulcer agent and signals through the protective PGE2, EP2, EP3, and EP4 receptors. It increases the cytoprotective levels of PGE2 necessary for maintaining integrity of the gastric mucosa. The risk for nonsteroidal anti-inflammatory drug (NSAID)-induced gastric or duodenal ulcer is decreased with concomitant use of misoprostol. Misoprostol can affect gastric, esophageal and duodenal mucosal integrity from insults related to acids and exodenous factors like NSAIDS. NSAIDs inhibit prostaglandin synthesis, thereby reducing mucus production, bicarbonate secretion, and mucosal blood flow. Continuous blood flow through the microvessels is very crucial for the function and maintenance of structural integrity of the gastrointestinal tract. Microcirculation delivers oxygen and nutrients to all tissues and cells and removes ad hoc generated toxic metabolites.

Ahluwalia et al. (2014) attempted to better understand the roles of PGEs and their receptors, signaling pathways including cAMP and CREB, and their relation to VEGF and angiogenesis in the tissue injury healing process (i.e. gastroduodenal and dermal ulcers). Using an esophageal ulcer model in rats, they demonstrated that esophageal mucosa expresses predominantly EP2 receptors and that esophageal ulceration triggers an increase in expression of the EP2 receptor, activation of CREB (the downstream target of the cAMP signaling), and enhanced VEGF gene expression. Treatment of rats with misoprostol, a PGE 1 analog capable of activating EP receptors, enhanced phosphorylation of CREB, stimulated VEGF expression and angiogenesis, and accelerated esophageal ulcer healing. In cultured human esophageal epithelial (HET-1A) cells, misoprostol increased intracellular cAMP levels (by 163-fold), induced phosphorylation of CREB, and stimulated VEGF expression. A cAMP analog (Sp-cAMP) mimicked, whereas an inhibitor of AMP-dependent protein kinase A (Rp-cAMP) blocked, these effects of misoprostol. These results indicate that the EP2/cAMP/protein kinase A pathway mediates the stimulatory effect of PGEs on angiogenesis essential for tissue injury healing via the induction of CREB activity and VEGF expression (Ahluwalia A, et al. AJP Gastrointest Liver Physiol. 2014 Sep. 15; 307(6):G602-G610).

### Preclinical Data Supporting the Use of Misoprostol in CDI

Data from a mouse model (of CDI) experiments have shown that misoprostol can be effective in helping to prevent recurrence of CDI infection and improve outcomes of patients with CDI (Data is shown in Example 1 above). Mice first treated with cefoperazone and then challenged by inoculation with spores of C. difficile fared significantly better when treated with misoprostol compared to placebo on multiple outcome variables including survival, diarrhea severity and intestinal permeability.

### Experimental Study Design

A total of 440 patients meeting enrollment criteria (listed below) are enrolled across 3 sites. The total study time period for study procedures followed by clinical monitoring is about 24 months (biomarker assays and other analyses can be completed after the 24-month time period). All participants receive the standard of care (oral vancomycin) under the care of their physician. After consenting to participate in the study, participants are randomized to receive either misoprostol (200 mcg po BID) or matching placebo for 14 days. Participants are monitored for a total time-period of approximately 10 weeks with the goal of monitoring for recurrence of CDI during an 8-week follow-up period from the time that the course of vancomycin is completed. Patients have blood and stool samples collected throughout the study (as described below) to assess adherence, biomarkers, and to confirm recurrence of CDI if necessary.

### Inclusion Criteria

1. Primary episode of CDI, defined as ALL of the following:
   a. 3 unformed (loose or watery) stools with a 24-hour period;
   b. A documented positive C. difficile toxin assay (enzyme immunoassay [EIA] or cellular cytotoxicity assay) or DNA PCR assay for toxigenic C. difficile from a stool sample collected while the subject was symptomatic; and
   c. No other explanation for diarrhea (e.g. laxatives).
2. At the time of randomization, having been treated for the primary episode of CDI with a single course of oral vancomycin for a total of at least 7 days, but not more than 10 days.
3 Be 50 years of age and able to provide signed and dated informed consent.

### Exclusion Criteria

1. Have had more than 1 episode of CDI within 6 months before the day of enrollment, as evidenced by self-reporting and EHR verification.
2. Have not recovered from primary episode of CDI by day 7 of vancomycin therapy, defined as ALL of the following:
   a. 3 unformed (loose or watery) stools per day for at least 2 consecutive days prior to and continuing to the time of randomization, and
   b. Abdominal discomfort must be absent or mild for at least 2 consecutive days prior to and continuing to the time of randomization.
3. Have received, or plans to use, any of the following for treatment of the primary episode of CDI:
   a. Any immunotherapy (e.g., intravenous immunoglobulin, bezlotoxumab).
   b. Any toxin-binding therapy (e.g., cholestyramine [Questran], colestipol [Colestid], or colesevelam [Welchol]).
4. Current or planned treatment with prostanoid therapy.
5. Diarrhea caused by another infection or underlying gastrointestinal disorder.
6. Have an acute febrile illness (fever >38° C. [100.4° F.]) on the day prior to the first dose of study drug.
7. Plan to receive an oral or parenteral (e.g., intravenous, intramuscular, or intraperitoneal) antibacterial therapy after randomization.
8 Have any contraindication to oral/enteral therapy (e.g., severe nausea/vomiting or ileus).
9. Have an absolute neutrophil count <1000/mm³ [1.0×10⁹/L] at screening.
10. Myocardial infarction within the past 6 months of enrollment.
11. Have a known immunodeficiency disorder, including but not limited to:
   a. HIV infection with CD4 count <200 or CD4 count of any level and not on highly active antiretroviral therapy
   b. Receiving, or plans to receive, treatment with systemic corticosteroids.
   c. Receiving, or plans to receive, myelosuppressive cancer chemotherapy.
12. Require or have an anticipated need for mechanical ventilation of vasopressors for hemodynamic support during the study.
13. Pregnant, nursing, or planning to become pregnant.
14. Have taken investigational drugs within 30 days before misoprostol administration.
15. Inability to understand the requirements of the study, inability to abide by the study restrictions and to return for the required treatments and assessments.
16. Have any clinically significant medical or surgical condition that in the investigator's opinion could interfere with the administration of study drug, interpretation of study results, or compromise the safety or well-being of the subject.
17. Known hypersensitivity to misoprostol.
18. Be unwilling or unable to follow study procedures (e.g., study visits, provide stool samples and undergo phlebotomy according to the study schedule, and reliably report information by phone), or not have a caregiver who can ensure that study procedures are followed.
19. If female, be pre-menopausal (cessation of menses greater than or equal to 1 year) and not surgically sterile or not following acceptable non-hormonal method of birth control such as abstinence, intrauterine device, or barrier control for at least 1 complete menstrual cycle before the screening visit, or using estrogen/progestin containing products for at least 2 months before the screening visit through discharge from the study.
20. Not have reliable access to telephone service to allow for contact with study personnel.
21. Be unable to be seen for routine clinical care either as an outpatient or inpatient at one of the three study sites.

To evaluate the exploratory objectives of the study assessing oral misoprostol compared to placebo in patients aged 50 years or older with a primary episode of CDI, the following endpoints are measured:
Primary endpoint: Rate of clinical recurrence of CDI in patients aged 50 years or older during the first 8 weeks after vancomycin therapy is stopped. Rate of recurrence among misoprostol treated participants is compared with that of placebo treated participants to determine efficacy of misoprostol in preventing rCDI. rCDI is defined as people who meet criteria for CDI in the 8-week follow-up period.
Safety endpoint: Safety and tolerability of misoprostol in patients aged 50 years or older with rCDI compared to placebo as measured by treatment emergent adverse events and standard laboratory assessments.
Secondary endpoint: Serum biomarkers/toxin antibodies (from blood samples}.
Secondary endpoint: Recovery of bowel microbiota (from microbiome collection.
Secondary endpoint: Number of recurrences during the follow-up period (for those who have a first recurrence during the follow-up time period.
Secondary endpoint: Time to resolution of diarrhea (TTROD; for those with recurrence}.
Secondary endpoint: Titer of toxin in stool (for those with recurrence}.
Secondary endpoint: Severity of disease (for those with recurrence}, determined using a combination of severity criteria used in hospital specific guidelines, SHEA/IDSA guidelines, Zar criteria, or other sources:
   WBC count greater than or equal to 15,000 cells/mm³ or >20,000 cells/mm³
   Serum creatinine 2:1.5×baseline
   Temperature >38.3° C.
   Albumin level <2.5 g/dL ICU admission
   Endoscopically or histologically confirmed pseudomembranous colitis
   Toxic megacolon, perforation, colectomy, or septic shock requiring ICU admission and pressors
   Hospital readmission
   Biomarkers in stool: Lactoferrin, Calprotectin, Cytokines (Boone J H, et al. Eur J Clin Microbiol Infect Dis. 2014 June; 33(6):1045-1051; Kim J, et al. Ann Lab Med. 2017; 37(1):53; El Feghaly R E, et al. Clin Infect Dis. 2013 Jun. 15; 56(12):1713-1721).
   IBS questionnaire or other instruments with quality of life questions

Subjects are in the study for 70 days. The total duration of the study is 24 months.

The study described in this example is used to: evaluate the safety and tolerability of misoprostol dosed orally for up to 14 days in older adults (50 years of age) being treated with the standard of care for CDI, measured by treatment emergent adverse events and standard laboratory assessments; evaluate the efficacy of misoprostol for prevention of rCDI, by determining if misoprostol can modify the rate and severity of rCDI in adults 50 years of age during the first 8 weeks after standard oral vancomycin therapy is completed; and determine the effect of misoprostol on the gut microbiome and diarrhea occurrence/severity.

### Misoprostol

The study drug, misoprostol, is purchased from Novel Laboratories, Inc. Misoprostol is over-encapsulated using #00 purple locking capsules. The tablet is covered with microcrystalline cellulose to fill the capsule. The matching placebo manufactured for this study is identical in appearance.

Misoprostol contains equal amounts of the following:

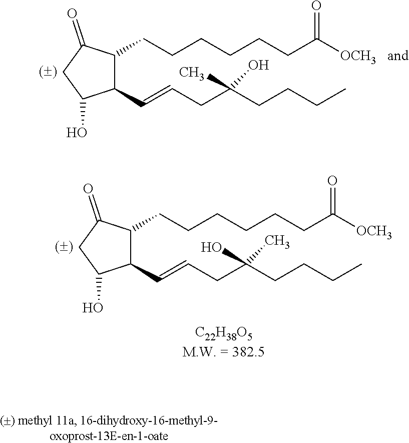

### Doses and Mode of Administration

Patients randomized to receive misoprostol take two 200 μg capsules by mouth per day (200 mcg po BID) for 14 days. Patients randomized to placebo take two matching placebo capsules by mouth per day for 14 days.

### Study Procedures

This example discloses a randomized, double-blind, placebo-controlled, study in patients age 50 years of age or older who have been recently diagnosed with and treated for a primary episode of CDI with oral vancomycin. The study is discussed with patients presenting with a primary episode of CDI, which is based on the presence of diarrhea (three unformed stools or more in 24 hours), the presence of C. difficile toxin A or B in stool samples collected while the subject was symptomatic, and the absence of another CDI diagnosis in the preceding 6 months. Patients whose symptoms have resolved by day 7 of oral vancomycin are eligible for randomization and continued participation in the study. At day 7, participants are randomized to receive misoprostol or matching placebo. Concomitant treatment with other potentially effective treatments for CDI is not allowed. All enrolled patients continue to take the standard of care oral vancomycin (typically 125 mg four times a day but can be a higher dose as determined by the treating physician) for an additional 4 days after starting misoprostol treatment (such that all participants have a 4 day overlap of vancomycin and misoprostol treatment), and begin taking an investigational 200 μg oral misoprostol or placebo two times a day for 14 days. Dosing of misoprostol or placebo starts on day 7-10 of vancomycin treatment upon confirmation of clinical resolution of diarrhea, and resolution of serious CDI signs and symptoms (resolution typically occurs on or near day 3 on vancomycin therapy). Patients who fail to respond to vancomycin therapy by day 7 of treatment are withdrawn from the study (data and stool samples are saved for analysis). Patients are randomized 1:1 between the misoprostol and placebo arms of the study. All patients are monitored for recurrence via phone call once per week for an additional 7 weeks (the total duration of study participation is 10 weeks).

In the event that a participant thinks he/she is having a recurrence of CDI the participant comes to a study site for a study visit to collect a stool sample and the sample is tested for the presence of C. difficile toxin A or B. If the participant has a confirmed recurrence (positive toxin test) he/she continues to be monitored for the duration of the 10-week study period; however, no additional study interventions are performed on the participant. The participant's care subsequent to a recurrence is the SOC at the discretion of his/her physician (he/she stops taking misoprostol/placebo if recurrence occurs during misoprostol/placebo treatment).

Throughout the study, participants are followed as either inpatients or outpatients, depending on their individual clinical conditions and routine care context. Safety is monitored through the recording of adverse events, clinically relevant changes in physical examinations, and clinical safety laboratory testing when adverse events are reported. A diagnostic stool sample (or rectal swab samples if stool sample cannot be produced) is collected to confirm CDI infection at periodic study visits, including (1) the study visit at day 7-10, (2) completion of investigational therapy (for future microbiome sequencing), (3) in the case of a possible recurrence (for toxin testing), and (4) at the end of study period. Blood draws occur at periodic study visits, including (1) the study visit at day 7-10 (to obtain baseline data before treatment with study drug or placebo), (2) near completion of investigational therapy to assess adherence and to test for antibodies and other biomarkers), (3) in the case of a possible recurrence (to test for antibodies and other biomarkers), and (4) at the end of study period (to test for antibodies and other biomarkers).

Pre-enrollment period (Days 0-3): Patients with suspected CDI diagnosis (parameters for primary episode of CDI as outlined above) are approached to discuss the study and enrollment. It is not possible to consent patients until research staff have obtained confirmation of a positive CDI laboratory test. Patients begin their physician-directed standard treatment for a primary episode of CDI, i.e. oral vancomycin 125 mg oral QID. Stool samples are held such that stool samples from patients with confirmed CDI infection (defined in inclusion criteria) who give informed consent and enroll in the study can be saved for future analyses.

Study Enrollment (Days 3-7): Patients with confirmed CDI diagnosis (parameters for primary episode of CDI as outlined above) are contacted (by phone if outpatient) to discuss the study and enrollment. Study personnel obtain informed consent (by e-consent if possible for outpatients) and enroll participants.

Study Visit and Randomization (Day 7-10): Upon confirmation that the patient has responded to oral vancomycin treatment (e.g., resolution of symptoms as outlined in inclusion criterion #4), the patient is randomized to misoprostol or placebo. The study participant begins taking investigational therapy in addition to oral vancomycin. Participant continues taking oral vancomycin such that there is a 4-day overlap during which time the participant is taking both oral vancomycin and investigational therapy.

Study Drug Administration Period (A 14-day period during days 7-24) and Follow-up through Day 70: Participants are instructed to record date and time of each study drug administration, oral temperature, all adverse events, date and time of any unformed (loose or watery) stools, and medications. In addition, study personnel contact participants at least once weekly through Day 70 (at scheduled study visits or via telephone contacts) to inquire about adherence to study drug dosing (when applicable), proper completion of the study questionnaires, and in particular, any occurrence of diarrhea and/or loose/watery stools.

TABLE 1
Schedule of Events for Misoprostol Study

| | Day 0 | Day 2-3 | | | Day 21-70 | |
|---|---|---|---|---|---|---|
| | Pre-enrollment | Phone call and | Day 7-10 | Day 21-24 | *Study visit if | Day 70 |
| Assessment | period | consent | Study Visit | Study Visit | recurrence | Study visit |
| Confirm positive CDI toxin test | | X | | | | |
| Discuss study with CDI patients to | | X | | | | |
| determine eligibility | | | | | | |
| Start SOC (not research) | X | | | | | |
| Start study drug (duration of 14 | | | X | | | |
| days) | | | | | | |
| Informed Consent | | X | | | | |
| Confirm CDI symptom resolution | | | X | | | |
| Symptom questionnaire | | X | X | | | |
| Concomitant meds | | X | X | X | X | X |
| Monitor for symptoms | | | X | X | X | X |
| CDI toxin test | | | | | X* | |
| Blood Draw | | | X | X | | X |
| Stool sample/swab (save SOC test | X | | | X | X* | X |
| sample | | | | | | |
| Phone call check in (1x per week) | | X | X | X | X | X |
| Adverse events | | | X | X | X | X |

Misoprostol may have gastrointestinal (GI) side effects (e.g., diarrhea, abdominal cramping) during the first few days of use, which is not to be confused with recurrence of disease. Since patients do not generally experience a recurrence until after they have completed the full course of vancomycin therapy, any GI symptoms occurring during the first 4 days of investigational therapy (which overlaps with vancomycin treatment) are presumed to be a side effect of misoprostol (captured as an adverse event) and not a recurrence of CDI. Depending on the severity of symptoms (specifically GI symptoms such as diarrhea and abdominal cramping), misoprostol dose may be reduced to minimize discomfort (for example, 100 mcg po BID minimum dose).

For data summary, frequency tables are generated for categorical variables, and continuous variables are expressed as means±SD. Normal distribution of data is assessed using the Kolmogorov-Smimov test. Comparisons between control and treatment patients are analyzed using ANOVA, t-test or Kruskal-Wallis test for continuous variables and Fisher exact tests for categorical variables. The outcomes of interest are studied that differ between the patient groups who receive treatment vs. placebo. The primary outcome of this study is the clinical recurrence rate of CDI in patients aged 50 years or older during first 8 weeks after SOC vancomycin therapy is completed. Secondary outcomes include treatment emergent adverse events (AE), standard laboratory assessment for AE, recovery of bowel microflora, time to recurrence, time to resolution of diarrhea, severity of diarrhea, titer of toxin in stool. Serum biomarkers/toxin antibodies from blood draws are recorded for each patient. A two-tailed p<0.05 is considered significant. The sample size calculation is based on treating the infection recurrence rate as a proportional measurement of recurrent events compared between the placebo and misoprostol treatment group following vancomycin therapy.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A method for treating or preventing the recurrence of a C. difficile infection comprising administering to a host in need thereof an effective amount of misoprostol or a pharmaceutically acceptable salt thereof.

2. The method of claim 1, wherein the misoprostol dose is 200 μg.

3. The method of claim 1, wherein the misoprostol is administered four times daily.

4. The method of claim 1, wherein the misoprostol is administered two times daily.

5. The method of claim 1, wherein the misoprostol is administered for two weeks.

6. The method of claim 1, wherein the misoprostol is in a tablet dosage form.

7. The method of claim 1, wherein the misoprostol is comprised in an extended release dosage form.

8. The method of claim 1, wherein the misoprostol is released directly to the colon.

9. A method for treating or preventing the recurrence of a C. difficile infection comprising administering to a host in need thereof an effective amount of misoprostol, or a pharmaceutically acceptable salt thereof, and an antibiotic.

10. The method of claim 9, wherein the antibiotic is selected from the group consisting of fidaxomicin, metronidazole, vancomycin, cadazolid, CRS3123, SMT19969, surotomycin, teicoplanin, tigecycline, ramoplanin, NVB302, and nitazoxanide.

11.-14. (canceled)

15. The method of claim 9, wherein the misoprostol dose is 200 μg.

16.-18. (canceled)

19. The method of claim 9, wherein the misoprostol is in a tablet dosage form.

20. The method of claim 9, wherein the misoprostol is comprised in an extended release dosage form.

21.-24. (canceled)

25. The method of claim 9, wherein the antibiotic is in a tablet dosage form.

26. The method of claim 9, wherein the antibiotic is comprised in an extended release dosage form.

27. (canceled)

28. The method of claim 9, wherein the misoprostol and the antibiotic are in a single tablet dosage form.

29. The method claim 9, wherein the antibiotic is vancomycin.

30. The method of claim 29, wherein the vancomycin dose is 125 mg.

31. A method for treating or preventing the recurrence of a C. difficile infection comprising administering to a host in need thereof an effective amount of misoprostol, or a pharmaceutically acceptable salt thereof, and a probiotic.

32. The method of claim 31, wherein the probiotic is selected from the group consisting of Lactobacillus, Bifidobacterium, Non-difficile Clostridium, Non-toxigenic Clostridium difficile, Saccharomyces, and Streptococcus.

33.-70. (canceled)
